(19) United States
(12) Patent Application Publication
(10) Pub. No.: US 2017/0196819 A1
(43) Pub. Date: Jul. 13, 2017

(54) PHARMACOTHERAPEUTIC BIOADHESIVE COMPOSITION

(71) Applicant: The Texas A&M University System, College Station, TX (US)

(72) Inventor: Xiaohua Liu, Plano, TX (US)

(21) Appl. No.: 15/402,212

(22) Filed: Jan. 9, 2017

Related U.S. Application Data
(60) Provisional application No. 62/276,842, filed on Jan. 9, 2016.

(51) Int. Cl. A61K 9/70; A61K 9/00; A61K 47/34; B29C 39/36; A61K 47/32; A61K 47/36; A61K 47/10; B29C 39/00; A61K 31/573; A61K 47/38

(52) U.S. Cl. CPC A61K 9/7007; A61K 31/573; A61K 9/006; A61K 47/34; A61K 47/38; A61K 47/32; A61K 47/36; A61K 47/10; B29C 39/003; B29C 39/36

(57) ABSTRACT

Embodiments of the claimed invention are directed to a biodegradable mucosal bioadhesive composition that delivers a therapeutic agent specifically to the target site, without loss to saliva or absorption by non-targeted areas. In certain embodiments, the outer layer of the composition also protects the covered area from physical contact.

### CROSS-REFERENCE TO RELATED APPLICATIONS

This application claims the benefit of priority to and incorporates by reference the entire disclosure of U.S. Provisional Patent Application No. 62/276,842 filed on Jan. 9, 2016.

### STATEMENT REGARDING FEDERALLY SPONSORED RESEARCH AND DEVELOPMENT

This invention was made with government support under Grant No. 1R41DE024343-01 awarded by the National Institutes of Health. The government has certain rights in the invention.

### BACKGROUND OF THE INVENTION

Oral ulceration is one of the most commonly encountered oral diseases, with an estimated prevalence of 4% in the USA. Aphthous ulcers may affect as many as 25% of the population worldwide. Ulceration of the oral mucosa can cause soreness that affects individual's eating, drinking, and speaking, and ulcers of long-duration can even cause malnutrition that severely affects quality of life. Clinically, topical treatment with corticosteroids remains the first-line clinical therapy for oral ulcerations, and corticosteroids are delivered through a gel, oral base (paste) or rinses which provide temporary symptomatic relief. However, the effectiveness of current therapies is often greatly reduced by the fact that the medication can be diluted or eliminated within minutes by the patient's saliva, or through rubbing against the tongue or other parts of the mouth. Furthermore, continual usage of the amounts of the corticosteroid gel, paste, or rinse leads to potential systemic effect and significant local side effects, such as oral candidiasis, a yeast infection which further exacerbates the patient's soreness. While reports of oral mucoadhesive devices to deliver corticosteroids have appeared in the literature, these devices deliver the steroid in a multidirectional and uncontrollable manner thus potentially decreasing the local effective dose.

### SUMMARY OF THE INVENTION

The biodegradable mucosal bioadhesive composition of the claimed invention comprises polylactic acid, sodium carboxymethyl cellulose, carbopol, chitosan, PEG, and a therapeutic agent, capable of unidirectional drug delivery utilizing a three-sided sealing layer. A temporarily impermeable, biodegradable outer layer surrounds the internal layer containing the therapeutic agent, while both the inner and outer layers degrade over time as the therapeutic agent is delivered over time.

A method of fabricating a bioadhesive composition involving a series of casting mixtures and air-drying is utilized to form the bioadhesive compositions. The method typically comprises fabricating a well-defined hollow cylindrical device; casting a mixture of biodegradable poly(L-lactic acid) (PLLA) and bioadhesive polymer solution into the mold; allowing the mixture to air-dry to form the backing layer; dissolving the supporting material of the mold; casting the bioadhesive polymer aqueous solution blended with drugs on the backing layer; and air-drying the newly cast layer to form the drug-containing bioadhesive layer.

### BRIEF DESCRIPTION OF DRAWINGS

FIGS. 1A, 1B and 1C show the mechanical properties of a bioadhesive composition in accordance with an embodiment of the claimed invention; and

FIG. 2 shows the in vitro release profiles of clobetasol propionate from the bioadhesive composition in accordance with an embodiment of the claimed invention.

### DESCRIPTION OF EXEMPLARY EMBODIMENTS

Embodiments of the claimed invention are directed to a biodegradable mucosal bioadhesive composition. In certain embodiments, the bioadhesive composition comprises one or more of polylactic acid, sodium carboxymethyl cellulose, carbopol, chitosan, and PEG (Polyethylene glycol).

In certain embodiments, the bioadhesive composition utilizes a three-sided sealing layer for the bioadhesive composition, wherein the three-sided sealing layer ensures unidirectional and well-controlled drug delivery capability, eliminating potential leakage.

In certain embodiments, the bioadhesive composition also serves as a temporary protective barrier for the covered area from friction or adjacent structures.

In certain embodiments, the bioadhesive composition is designed to deliver a therapeutic agent specifically to the target site, without loss to saliva or absorption by non-targeted areas. In certain embodiments, the bioadhesive's specificity is ensured through the use of a temporarily impermeable, biodegradable outer layer that surrounds an internal layer containing the therapeutic agent. In an embodiment of the invention, the therapeutic agent is clobetasol propionate

In an embodiment of the invention, the bioadhesive composition shows a more gradual release of a therapeutic agent compared to the gel counterpart. FIG. 2 shows the in vitro release profiles of clobetasol propionate (CP) from the bioadhesive patch and a commercial CP gel (0.05%). The bioadhesive layer of the patch had a HPMC/Carbopol polymer ratio of ¼.

The bioadhesive composition is composed of a sealing outer layer and an inner bioadhesive drug layer. In an embodiment of the invention, poly(L-lactic acid) is used to prepare the sealing layer. In an embodiment of the invention, a mixture of hydroxypropyl methylcellulose (HPMC) and a crosslinked polyacrylic acid polymer (CARBOPOL®) is used to fabricate the bioadhesive layer. In some embodiments, the composition may comprise one or more of the following: sodium alginate, gelatin, pectin, Poly(vinyl alcohol), Poly(acrylic acid), Poly(ethylene oxide), Poly(vinyl pyrrolidone), methylcellulose, methylethyl cellulose, gum tragacanth, and soluble starch.

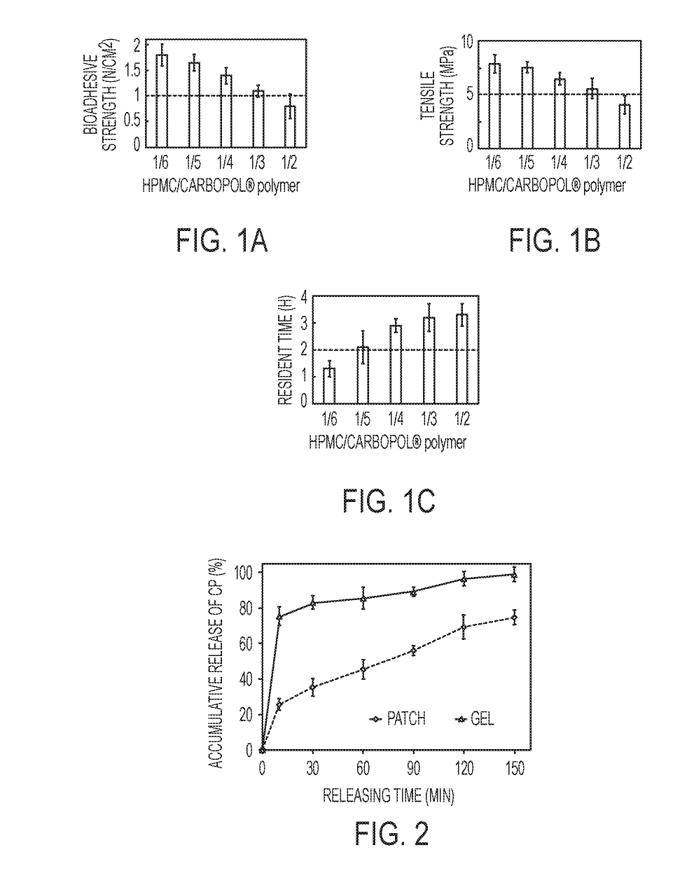

FIGS. 1A, 1B and 1C show the mechanical properties of the bioadhesive composition at different HPMC/Carbopol polymer ratios. FIG. 1A shows the bioadhesive strength of the composition as a function of the ratio of HPMC to crosslinked polyacrylic acid polymer in the inner layer; FIG. 1B shows the tensile strength of the composition as a function of the ratio of HPMC to crosslinked polyacrylic acid polymer in the inner layer; and FIG. 1C shows the resident time of the therapeutic in the composition as a function of the ratio of HPMC to crosslinked polyacrylic acid polymer in the inner layer. The patch was composed of a sealing layer and a bioadhesive drug layer. Poly(L-lactic acid) was used to prepare the sealing layer and HPMC/Carbopol were used to fabricate the bioadhesive layer. Nine samples of the bioadhesive patch for each identified ratio were used for the test.

The compositions having a lower ratio of HPMC to crosslinked polyacrylic acid polymer showed a greater tensile strength and bioadhesive strength. Conversely, the compositions having a higher ratio of HPMC to crosslinked polyacrylic acid polymer displayed a greater resident time of the therapeutic agent in the inner layer.

In certain embodiments, the therapeutic agent is directionally delivered towards the targeted site, while both the inner and outer layers degrade over time. Additionally, the outer, inert layer also protects the covered area from physical contact. In certain embodiments, the kinetics of degradation is such that the inner layer ceases delivering the therapeutic agent when the outer layer is degraded or dissolved.

Another embodiment of the invention is directed to a method of fabricating the adhesive composition. The method comprises fabricating a well-defined hollow cylindrical device frame using a reverse solid-free form fabrication technique. A mixture of biodegradable poly(L-Iactic acid) (PLLA) and bioadhesive polymer solution is cast into the mold, following which, the mixture is allowed to air-dry to form the backing layer. The bioadhesive polymer is added into the PLLA solution to enhance the adhesion between the backing layer and bioadhesive drug layer. The supporting material of the mold is dissolved and the bioadhesive polymer aqueous solution blended with drugs is cast on the backing layer. The newly cast layer is air-dried to form the drug-containing bioadhesive layer. Finally, a linear releasing siliconized film is utilized to cover the release surface of the formed bioadhesive composition.

In certain embodiments, the bioadhesive compositions may be either single cells of predetermined sizes, or may comprise multiple cells with predesigned boundaries for cutting. In further embodiments, the bioadhesive composition may make use of a honeycomb pattern of side barriers and drug delivery center.

While the present invention has been described in terms of certain preferred embodiments, it will be understood, of course, that the invention is not limited thereto since modifications may be made to those skilled in the art, particularly in light of the foregoing teachings.

What is claimed is:

1. A biodegradable mucosal bioadhesive composition comprising polylactic acid, sodium carboxymethyl cellulose, carbopol, chitosan, PEG, and a therapeutic agent.

2. The biodegradable mucosal bioadhesive composition according to claim 1, wherein the bioadhesive composition utilizes a three-sided sealing layer to ensure unidirectional and well-controlled drug delivery.

3. The biodegradable mucosal bioadhesive composition according to claim 2, wherein the bioadhesive composition delivers a therapeutic agent.

4. The biodegradable mucosal bioadhesive composition of claim 3, wherein the therapeutic agent is clobetasol propionate.

5. A method of fabricating a bioadhesive composition, the method comprising:
fabricating a hollow cylindrical device frame;
casting a mixture of biodegradable poly(L-lactic acid) (PLLA) and bioadhesive polymer solution into a mold;
allowing the mixture to air-dry to form a backing layer;
dissolving a supporting material of the mold;
casting a bioadhesive polymer aqueous solution blended with drugs on the backing layer; and
air-drying the newly cast layer to form a drug-containing bioadhesive layer.

6. The method according to claim 5 wherein the hollow cylindrical device frame is fabricated using a reverse solid-free form fabrication technique.

7. The method according to claim 5 wherein the bioadhesive polymer is added into the PLLA solution to enhance adhesion between the backing layer and bioadhesive drug layer.

8. The method according to claim 5 wherein drug-containing bioadesive layer is covered by a releasing linear siliconized film.

9. A biodegradable mucosal bioadhesive composition comprising a temporarily impermeable, biodegradable outer layer surrounding an internal layer containing a therapeutic agent, wherein delivery of the therapeutic agent is directionally targeted towards a specific site while both the inner and outer layers degrade over time.

10. The biodegradable mucosal bioadhesive composition of claim 9, wherein the outer layer ensures directional delivery of the therapeutic agent towards the targeted site without loss to saliva or absorption by non-targeted areas.

11. The biodegradable mucosal bioadhesive composition of claim 9, wherein the internal layer ceases delivering the therapeutic agent as the outer layer is degraded or dissolved.

12. The biodegradable mucosal bioadhesive composition of claim 9, wherein the bioadhesive composition may be of either single cells of predetermined sizes, or may comprise multiple cells with predesigned boundaries for cutting.

13. The biodegradable mucosal bioadhesive composition of claim 9, wherein the bioadhesive composition may make use of a honeycomb pattern of side barriers and drug delivery centers.
